(19) United States
(12) Patent Application Publication
(10) Pub. No.: US 2026/0249051 A1
(43) Pub. Date: Aug. 27, 2026

(54) INTRAVENOUS LINE MANAGEMENT SOLUTIONS

(71) Applicant: CareFusion 303, Inc., San Diego, CA (US)

(72) Inventors: Shishir PRASAD, Ramsey, NJ (US); Adel SHAMS, Verona, NJ (US); Manish KUMAR, Bengaluru (IN); Praveen NALAWADE, Bengaluru (IN); Karthik MR, Bengaluru (IN); Mohammed Mehtab KHAN, Bengaluru (IN); Balaji KANNAN, Chennai (IN); William Thomas KELLY, White Plains, NY (US); Ajay Vijay Suryavanshi, Pune (IN); Shangxian Zhu, Brookfield, WI (US); Lydia Marie LEHMAN, Fairfax, VA (US)

(21) Appl. No.: 19/061,815

(22) Filed: Feb. 24, 2025

(51) Int. Cl. A61M 25/02; A61M 5/14

(52) U.S. Cl. CPC A61M 25/02; A61M 5/1413; A61M 2025/024; A61M 2205/6072

(57) ABSTRACT

Intravenous (IV) line attachments and IV line attachment racks can be used to efficiently manage and organize multiple intravenous (IV) lines. Each line attachment has an attachment mechanism for receiving an IV line, a coupling mechanism for coupling the line attachment to neighboring line attachments, and an indicator. The line attachments on a given IV line have matching indicators, which help caregivers identify each IV line. Similarly, each line attachment rack has a plurality of attachment mechanisms. In some embodiments, the line attachment rack can transition between a collapsed configuration and an expanded configuration. In the expanded configuration, the line attachment rack might have additional attachment mechanisms or spread out the existing attachment mechanisms. An IV line can be attached to at least two line attachment racks via at least two attachment mechanisms. The at least two attachment mechanisms to which the IV line is attached have matching indicators.

### TECHNICAL FIELD

The present invention relates generally to fluid delivery devices and systems and, in particular, to intravenous (IV) line management systems.

### BACKGROUND

Medical treatments often include fluid delivery through the infusion of a medical fluid (e.g., a saline solution or a liquid medication) to patients using an intravenous (IV) catheter. In situations where a patient receives fluids from multiple IV catheters and is, thus, connected to multiple IV lines, caregivers can find it difficult to manage all of the IV lines. However, the caregivers'ability to manage a set of IV lines properly is crucial—especially for high-acuity patients in intensive care units or operating rooms—to reduce the risk of fluid delivery errors.

Traditionally, caregivers have managed IV lines by coupling handwritten labels to the IV lines, spreading out the IV sets to separate the IV lines, and trying to align IV catheters and IV bags with their corresponding infusion pumps and channels.

### SUMMARY

The conventional methods of managing multiple IV sets have inefficiencies that increase the occurrence of confusion and improper fluid delivery. For example, while coupling a handwritten label to an IV line can help caregivers identify the fluid being delivered by the IV line, caregivers need to update and change these labels on a regular basis. Not only does this require a lot of time from caregivers, but this also relies on consistent and accurate manual recordation, which is unlikely in high-intensity situations. Moreover, a patient might have multiple caregivers who take care of him during different shifts, and information can get lost between different caregivers as they change shifts. Additionally, a handwritten label on an IV line still requires a caregiver to manually trace the IV line to match an IV bag or infusion pump on one side of the IV line to an IV catheter on the other.

Moreover, while spreading out the IV sets helps separate the IV lines and limits tangling, caregivers do not always have enough space to spread out the IV sets. Space in hospitals, operating rooms, homecare settings, etc. can be limited, which means that the IV sets need to be kept together.

Finally, aligning different components of an IV set can also help caregivers keep track of a plurality of IV lines, but maintaining this alignment is not always possible. To meet a patient's needs, caregivers often have to adjust, change, or introduce equipment, which might require the caregivers to move the IV sets. Additionally, the caregivers might also have to adjust or replace the IV sets themselves.

Therefore, it is desirable to have an IV line management system that can help caregivers organize information about the fluids being delivered by multiple IV sets as well as organizing the IV sets themselves. This can be accomplished by attaching organization devices, such as the line attachments or line attachment racks described herein, to IV lines. The organization devices can be compatible with radio frequency identification tags that facilitate digital storage and access of information, which standardizes the process by which caregivers record and receive information about the IV sets with which they are working. The organization devices can also connect to each other, which connects the IV lines and reduces the occurrence of tangling. Finally, these organization devices can have indicators that enable caregivers to quickly trace an IV line (e.g., to confirm that a catheter is delivering the correct fluid).

Accordingly, some embodiments of the present disclosure are directed to a management system for intravenous (IV) lines comprising at least one pair of line attachments, each line attachment of the at least one pair of line attachments being configured to attach to one of the IV lines and comprising: an inner hose comprising (a) a tube configured to receive the IV line and (b) a slot extending longitudinally along the tube, the inner hose being configured to flex open at the slot and wrap around the IV line; and an outer housing comprising (a) a hollow interior configured to receive the inner hose and (b) an outer surface comprising a protrusion and a pocket, wherein the protrusion of each line attachment is configured to be removably coupled to the pocket of another line attachment, and the pocket of each line attachment is configured to removably coupled to the protrusion of another line attachment.

In some embodiments, a first pair of line attachments is configured to attach to a first IV line of the IV lines and a second pair of line attachments is configured to attach to a second IV line of the IV lines. Optionally, both line attachments in the first pair of line attachments comprises a first color and both line attachments in the second pair of line attachments comprises a second color, the first color being different from the second color. Optionally, both line attachments in the first pair of line attachments comprises a first QR code and both line attachments in the second pair of line attachments comprises a second QR code, the first QR code being different from the second QR code.

In some embodiments, the inner hose comprises one or more indicators, and the outer housing comprises a window configured to surround one indicator of the one or more indicators when the inner hose is within the hollow interior of the outer housing.

In some embodiments, the protrusion of each line attachment comprises an application surface.

In some embodiments, a first line attachment of the at least one pair of line attachments is configured to be positioned next to a fluid source, and a second line attachment of the at least one pair of line attachments is configured to be positioned next to a patient receiving fluid from the fluid source.

In some embodiments, each line attachment comprises a capacitive sensor configured to identify a fluid in the IV line when the line attachment is attached to the IV line.

Other embodiments of the present disclosure are directed to a management system for intravenous (IV) lines comprising at least one pair of line attachments, each line attachment of the at least one pair of line attachments being configured to attach to one of the IV lines, each line attachment comprising: a receiving area comprising a top surface configured to receive the IV line, a bottom surface, a first side, and a second side opposite the first side; a strap movably coupled to a first side of the receiving area by a hinge, wherein the strap is configured to connect to the second side of the receiving area to couple the IV line to the line attachment when the IV line is in the receiving area; a plate protruding from either the first side or the second side of the receiving area; and rails protruding from the bottom surface of the receiving area, wherein the plate of each line attachment is configured to be removably coupled to the rails of another line attachment, and the rails of each line attachment is configured to removably coupled to the plate of another line attachment.

In some embodiments, the top surface of the receiving area comprises a groove, wherein the groove is configured to limit deformation of the IV line when the IV line is coupled to the line attachment by the strap.

In some embodiments, the strap comprises a ratchet surface and the second side of the receiving area comprises a pawl, the ratchet surface being configured to secure the IV line to the receiving area by engaging with the pawl.

In some embodiments, a first pair of line attachments is configured to attach to a first IV line of the IV lines and a second pair of line attachments is configured to attach to a second IV line of the IV lines. Optionally, both line attachments in the first pair of line attachments comprises a first color and both line attachments in the second pair of line attachments comprises a second color, the first color being different from the second color. Optionally, both line attachments in the first pair of line attachments comprises a first QR code and both line attachments in the second pair of line attachments comprises a second QR code, the first QR code being different from the second QR code.

In some embodiments, at least one line attachment in each pair of line attachments comprises a near field radio frequency identification (RFID) tag.

Additionally embodiments of the present disclosure are directed to a management system for intravenous (IV) lines comprising line attachment racks configured to receive the IV lines, each rack comprising: a plurality of grooves, each groove of the plurality of grooves being configured to receive one IV line of the IV lines; and at least one sub-rack slidably coupled to the rack, wherein the at least one sub-rack is positioned inside the rack when the rack is in a collapsed configuration and extends longitudinally from the rack when the rack is in an expanded configuration, wherein a first portion of grooves of the plurality of grooves extends laterally across a top surface of the rack and a second portion of the plurality of grooves extends laterally across a top surface the at least one sub-rack.

In some embodiments, the line attachment racks comprise a first rack and a second rack, and each groove on the first rack corresponds with another groove on the second rack, such that corresponding grooves are configured to receive a same IV line. Optionally, the line attachment racks further comprise a QR code or a color indicator adjacent to each groove, wherein the corresponding grooves each comprise matching QR codes or color indicators.

In some embodiments, a third portion of grooves of the plurality of grooves extends laterally across a bottom surface of the rack and a fourth portion of the plurality of grooves extends laterally across a bottom surface of the at least one sub-rack.

In some embodiments, the at least one sub-rack comprises a first sub-rack and a second sub-rack, wherein the first sub-rack is positioned inside the rack adjacent to the top surface of the rack when the rack is in the collapsed configuration and extends longitudinally from a first side of the rack when the rack is in the expanded configuration, wherein the second sub-rack is positioned inside the rack adjacent to a bottom surface of the rack when the rack is in the collapsed configuration and extends longitudinally from a second side of the rack when the rack is in the expanded configuration, the second side of the rack being opposite the first side.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates multiple IV sets coupled to a patient and organized with an IV line management system, in accordance with some embodiments described herein.

FIGS. 2A-2I illustrate a first example of the IV line attachment, in accordance with some embodiments disclosed herein.

FIGS. 3A-3D illustrate a second example of the IV line attachment, in accordance with some embodiments disclosed herein.

FIGS. 4A-4C illustrate a third example of the IV line attachment, in accordance with some embodiments disclosed herein.

FIGS. 5A-5E illustrate a fourth example of the IV line attachment, in accordance with some embodiments disclosed herein.

FIGS. 6A-6D illustrate a first example of the IV line attachment rack, in accordance with some embodiments disclosed herein.

FIGS. 7A-7C illustrate a second example of the IV line attachment rack, in accordance with some embodiments disclosed herein.

FIGS. 8A and 8B illustrate a third example of the IV line attachment rack, in accordance with some embodiments disclosed herein.

### DETAILED DESCRIPTION

In the following detailed description, numerous specific details are set forth to provide a full understanding of the subject technology. The subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

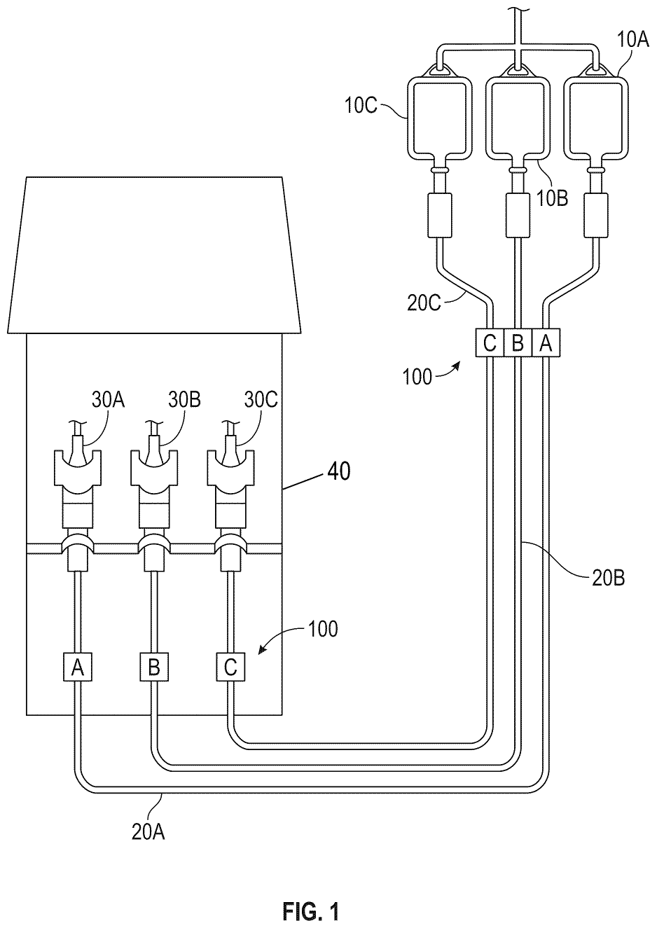

Referring now to the figures, FIG. 1 illustrates multiple IV sets coupled to a patient and organized with an IV line management system, in accordance with some embodiments described herein. Each IV set includes a medical fluid bag 10 (which can also be referred to as an IV bag 10 or a fluid source 10) filled with a fluid such as saline or medication. The fluid from the medical fluid bag 10 travels through the tubing 20 (which can also be referred to as the IV line 20) and the catheter 30 (or other fluid delivery device 30) into a vein in the patient's arm 40. FIG. 1 shows three IV sets A, B, and C, with IV set A having a medical fluid bag 10A, tubing 20A, and catheter 30A.

FIG. 1 also shows an IV line management system 100, which connects to the tubing 20 at two or more points. As described in greater detail below, the IV line management system 100 provides an organized system for labeling and tracking each IV line and separates individual IV lines to avoid tangling and delivery of improper fluids.

In some embodiments, the IV line management system 100 includes a near field radio frequency identification (RFID) tag. Traditionally, caregivers use handwritten labels to manage multiple IV sets. However, these handwritten labels are not standardized and can cause confusion, especially as a patient's caregivers change (e.g., different nurses handling the patient's medication over the course of several shifts). With RFID, the data associated with each IV set can be stored on a cloud interface and standardized, which keeps the IV sets more organized and reduces the likelihood of improper medication delivery. For example, caregivers can fill out data about the IV set in a standardized form or take and upload images of the medication labels, administration times, and infusion pump parameters. Additionally, the online database can be coupled with software that streamlines the caregivers'workflow. For instance, the online database can provide the caregivers with automated reminders for changing the IV tubing or the IV bag based on best practice guidelines. Optionally, the online database can be integrated with management analytics software (e.g., Becton Dickinson's Vascular Access Management) that analyzes the data stored on the cloud interface and reports findings to the caregivers. All of the IV line management systems disclosed herein are compatible with the RFID data storage system described.

Disclosed herein are IV line attachments, each of which can attach or connect to an IV line and couple to another IV line attachment, as well as IV line attachment racks, which are groups of IV line attachments.

### I. Intravenous Line Attachments

Some embodiments of the IV line management system disclosed herein are directed to IV line attachments. An IV line attachment (which are also referred to as line attachments) can have an attachment mechanism by which the line attachment is connected to an IV line (such as IV line 20 in FIG. 1). The attachment mechanism can comprise a flexible or elastic material that can be fastened to the IV line, a strap that secures the IV line to the line attachment, a groove into which the IV line can be snap fitted, etc. Essentially, the attachment mechanism is a fastener that a caregiver can use to attach the IV line to the line attachment quickly and conveniently. In some embodiments, the line attachment is permanently coupled to an IV line. In other embodiments, the line attachment can be removed from an IV line and used with a different IV line.

A line attachment can also have two or more coupling mechanisms that are configured to couple the line attachment to another line attachment. Every line attachment in a given IV line management system has the same coupling mechanisms, such that every line attachment can be connected to every other line attachment in the IV line management system. The uniform coupling mechanisms also facilitate connection of the line attachments in any order. In some embodiments, the coupling mechanisms comprise a male coupler and a female coupler (e.g., a key and a slot, a protrusion and a pocket, male and female snap-fit mechanisms, etc.). Other embodiments of the coupling mechanisms include adhesives, hook and loop connectors, and other suitable fasteners that facilitate easy, sleek, and efficient coupling of the line attachments to each other.

By using the coupling mechanisms to connect line attachments, a caregiver can effectively connect several IV lines, which can help the caregiver organize and keep track of the IV lines. Not only can connecting the line attachments reduce the likelihood of the IV lines becoming tangled, but connecting the line attachments can also prevent the caregiver from mixing up the fluids associated with the IV lines. For example, with the line attachments being connected, the caregiver can maintain a patient's IV lines in the exact same order, which reduces any ambiguity and confusion associated with identifying the fluid being delivered by each IV line. Additionally, a group of caregivers (e.g., nurses at a hospital who take turns taking care of the patient as they change shifts) can establish a standardized order for the IV lines and use the connected line attachments to maintain that standardized order. This can limit shuffling of the IV lines between various caregivers and reduce the likelihood that the incorrect fluid is delivered to the patient.

When using the IV line attachments as part of the IV line management system, each IV line will generally have at least two IV line attachments: one near the IV bag (such as IV bag 10 in FIG. 1) and one near the patient. This helps caregivers ensure that the proper fluid is being delivered to the patient (i.e., that the fluid from IV bag 10B in FIG. 1 is being delivered to the patient via IV line 20B and catheter 30B). IV line attachments that are on the same IV line can be referred to as corresponding line attachments. Corresponding line attachments can be marked with matching indicators to signify that they are attached to the same IV line. For example, referring to FIG. 1, the two line attachments on IV set A can both have a blue stripe, the two line attachments on IV set B can both have a red stripe, and the two line attachments on IV set C can both have a green stripe. A caregiver can use the indicators on any given IV line 20 to trace the IV line 20 between the IV bag 10 and the catheter 30 coupled to the patient. This can be useful because a busy caregiver can find it difficult to ensure that she is working with the correct IV set. With the indicators in place to help her quickly confirm, for instance, that the catheter 30B is connected to the IV bag 10B, the caregiver is less likely to remove catheter 30A from the patient's arm under the mistaken belief that catheter 30A is attached to IV bag 10B.

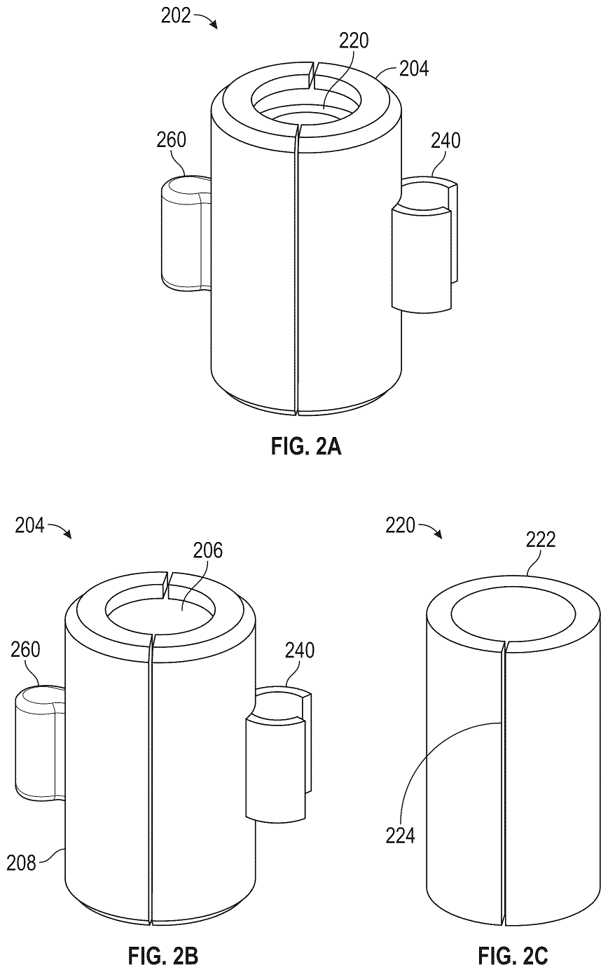

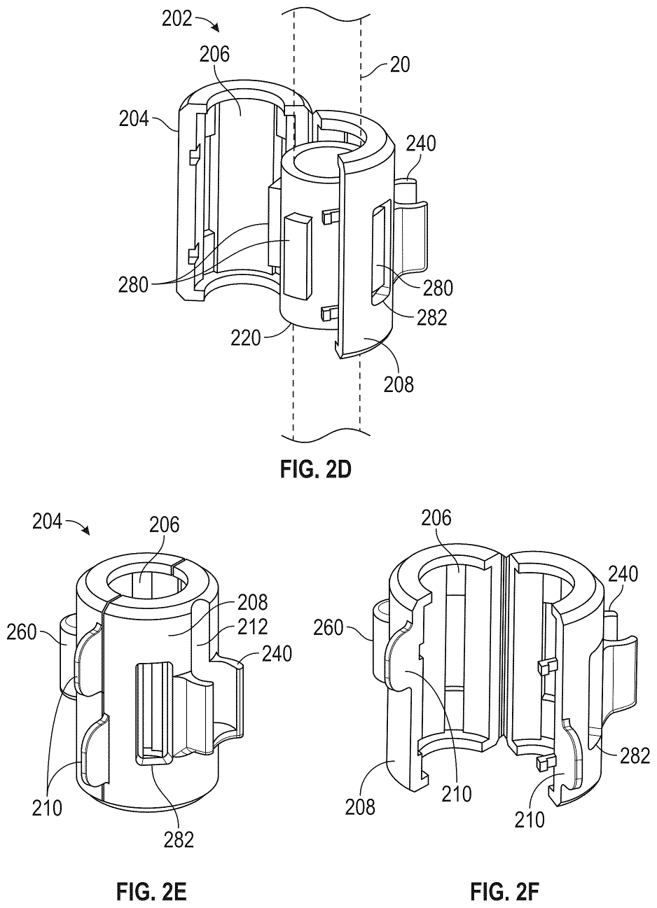

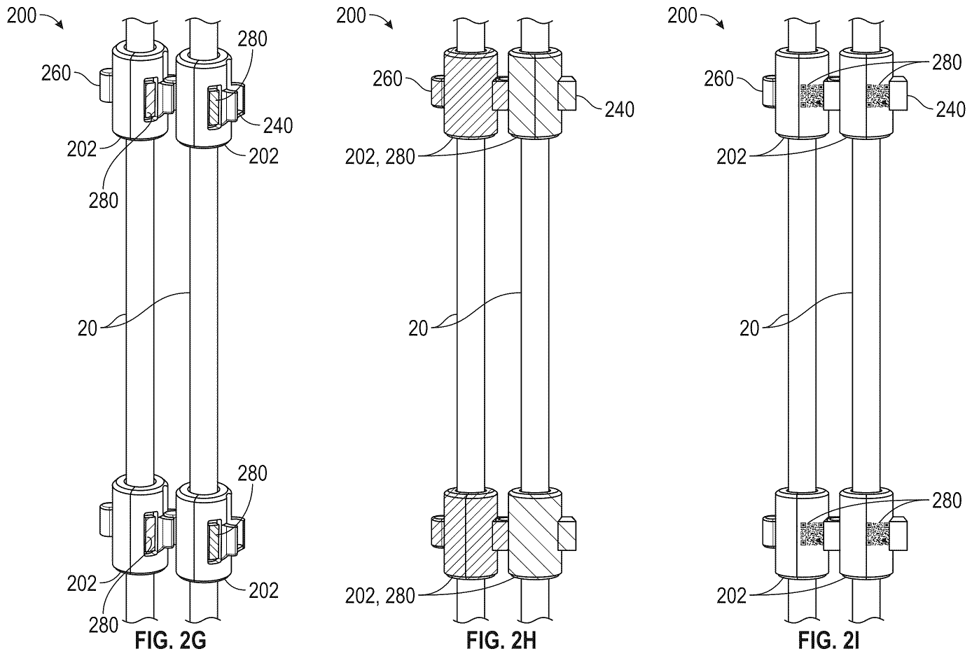

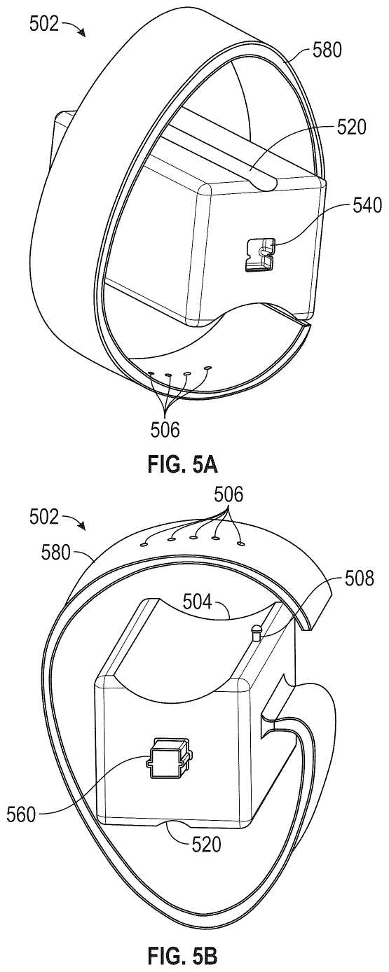

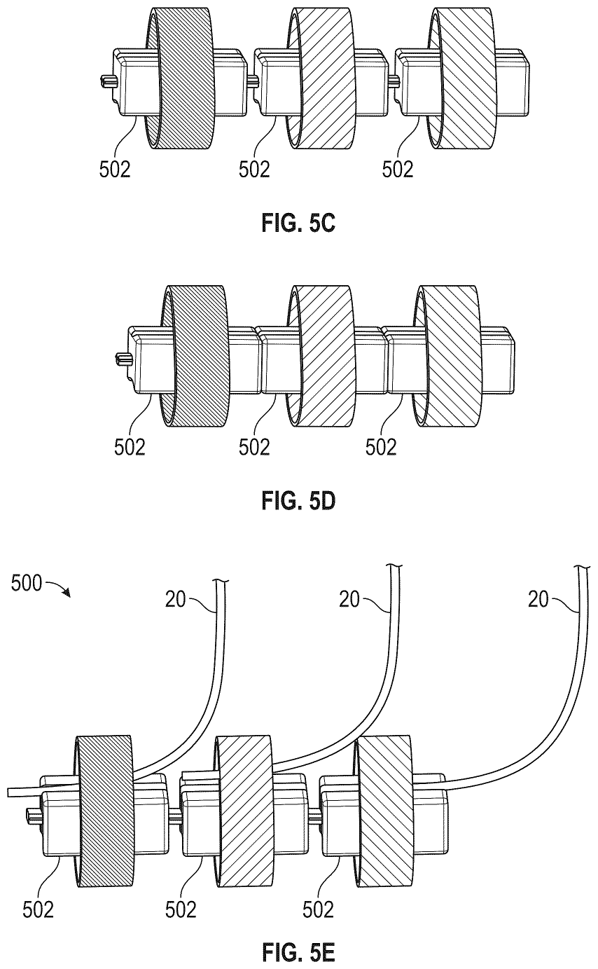

In some embodiments, the indicators are incorporated into the line attachments themselves. For example, corresponding line attachments can be the same color or be imprinted with the same number or letter. Other examples of the indicators being incorporated to the line attachments are described below with respect to the indicator 280 in FIGS. 2D-2F and the indicator 580 in FIGS. 5A-5E.

In other embodiments, the indicators are stickers or labels that can be applied to the line attachments. These indicators can comprise symbols, shapes, numbers, colors, QR codes, bar codes, etc. that are adhered to the outer surfaces of line attachments. Optionally, these indicators can comprise handwritten or typed labels with information about the type of medication, the infusion pump parameters, the medication administration times, etc.

FIGS. 2A-2I illustrate a first example of the IV line attachment, in accordance with some embodiments disclosed herein. As shown in FIGS. 2A-2C, this first example line attachment 202 has an inner hose 220 surrounded by an outer housing 204. The inner hose 220 (shown in FIG. 2C) serves as the attachment mechanism of the line attachment 202 and comprises a tube 222 with one slot 224 that cuts through the tube 222 and extends along the length of the tube 222. The inner hose 220 can be made of a flexible material, an elastomeric material, or any material with enough elasticity to facilitate opening of the inner hose 220 at the slot 224. This is because the inner hose 220 is configured to flex open at the slot 224 so that an IV line (such as IV line 20 in FIG. 1) can be inserted through the slot 224 and into the tube 222.

Once the inner hose 224 is wrapped around the IV line, the outer housing 204 is coupled to the inner hose 224 and the IV line. This can be seen in FIG. 2G. The outer housing 204 has a hollow interior 206 that is configured to receive the inner hose 220. The outer housing 204 can be made of rubber, an elastomer, or any soft, flexible material that can limit the discomfort experienced by patients in situation where the line attachment 202 is in contact with the patient.

In some embodiments, the outer housing 204 can be made of two halves that couple together. This can be seen in FIGS. 2D-2F. In some embodiments, the outer housing 204 is made of two completely separate halves that fit together by a snap fit mechanism, a slot and a key, a buckle, or other fastener. In other embodiments, the two halves of the outer housing 204 can be connected by a hinge. Optionally, the outer housing 204 has tabs 210 that a caregiver can use to separate the two halves and open the outer housing 204 to remove the line attachment 202 from the IV line (or to change the indicator 280 that is being displayed, which is described in greater detail below).

The outer surface 208 of the outer housing 204 has two coupling mechanisms. In the first example line attachment 202, the coupling mechanisms are the protrusion 260 and the pocket 240, which can be seen in FIGS. 2A, 2B, 2E, 2F, and 2G-2I. The protrusion 260 of each line attachment 202 is sized and shaped to fit inside the pocket 240 of another line attachment 202. Likewise, the pocket 240 of each line attachment 202 is sized and shaped to receive the protrusion 260 of another line attachment 202. The coupled line attachments 202 are depicted in FIGS. 2G-2I and described in greater detail below. In some embodiments, the protrusion 260 snap fits into the pocket 240. In other embodiments, the protrusion 260 is designed to slide into the pocket 240. In the embodiments where the protrusion 260 can slide into the pocket 240, the outer surface 208 of the outer housing 204 might have a slot 218 (shown in FIG. 2E) that is shaped to have the same contours of the protrusion 260.

FIGS. 2G-2I depict the line attachments 202 coupled to IV lines 20 as part of IV management systems 200. In particular, each of FIGS. 2G-2I depicts two coordinated pairs of line attachments 202, with one pair of line attachments 202 being connected to each IV line 20. FIGS. 2G-2I demonstrate that each line attachment 202 can be coupled to a neighboring line attachment 202 via coupling mechanisms 240 and 260.

The line attachments 202 can also have various types of indicators, such as the indicators described above. In some embodiments, the outer surface 208 of the outer housing 204 has a flat portion for easy application of the sticker or label indicators. In other embodiments, the protrusion 260 includes a flat surface or a plate that extends out of the outer surface 208 of the outer housing 204. In these embodiments, caregivers can apply the sticker or label indicators directly to the flat surface of the protrusion 260.

FIGS. 2D and 2E illustrate another type of indicator. In this embodiment, the indicators 280 are protrusions extending from the tube 222 of the inner hose 220. A window 282 is cut into the outer surface 208 of the outer housing 204 so that one of the indicators 280 can be seen from outside of the line attachment 202. As shown in FIG. 2D, the inner hose 220 has multiple indicators 280, but only one indicator 280 is visible through the window 282. Each indicator 280 can have a different color, pattern, number, barcode, etc., and the caregiver can select which indicator 280 to display in the window 282 by rotating the tube 222 relative to the hollow interior 206 of the outer housing 204.

FIGS. 2G-2I depict the line attachments 202 coupled to IV lines 20 as part of IV management systems 200. In particular, each of FIGS. 2G-2I depicts two coordinated pairs of line attachments 202, with one pair of line attachments 202 being connected to each IV line 20. Referencing FIG. 1, one line attachment can be positioned downstream near the catheter 30 in the patient's arm 40 or near the patient's arm 40, and the other line attachment can be positioned upstream near the IV bag 10 or an infusion pump. Each IV line 20 has a pair of line attachments 202 with coordinated indicators 280, such that both the downstream and the upstream line attachments 202 have an indicator 280.

FIG. 2G illustrates the protruded indicators 280 displayed in the windows 282 as described above with respect to FIGS. 2D and 2E. FIG. 2H shows solid colored line attachments 202, with each coordinated pair of line attachments 202 having the same solid color. In this embodiment, the indicators 280 are the solid colors, which can help caregivers trace the IV lines 20 faster because the solid color indicators 280 are visible from all angles. Optionally, the indicators 280 can be different patterns or symbols printed on the outer housings 204 of the line attachments 202. Finally, FIG. 2I shows that each line attachment 202 has an indicator 280 in the form of a QR code. Both line indicators 202 in a coordinated pair have the same QR code, and a caregiver can scan the QR codes to identify the coordinated pairs.

In some embodiments, each line attachment 202 comprises a sensor. The sensor can identify the fluid flowing through the IV line while the line attachment 202 is attached to the IV line. The sensor can communicate the fluid identification data with an infusion pump in order to crosscheck the fluid in the IV line. In some embodiments, the sensor is a capacitive sensor that identifies the capacitance or impedance value of the fluid and compares that data to data in a drug library.

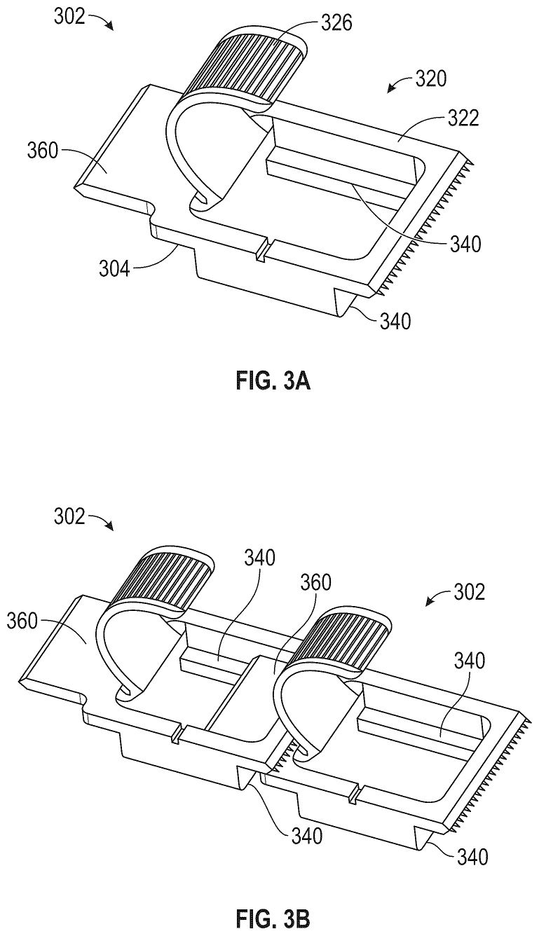

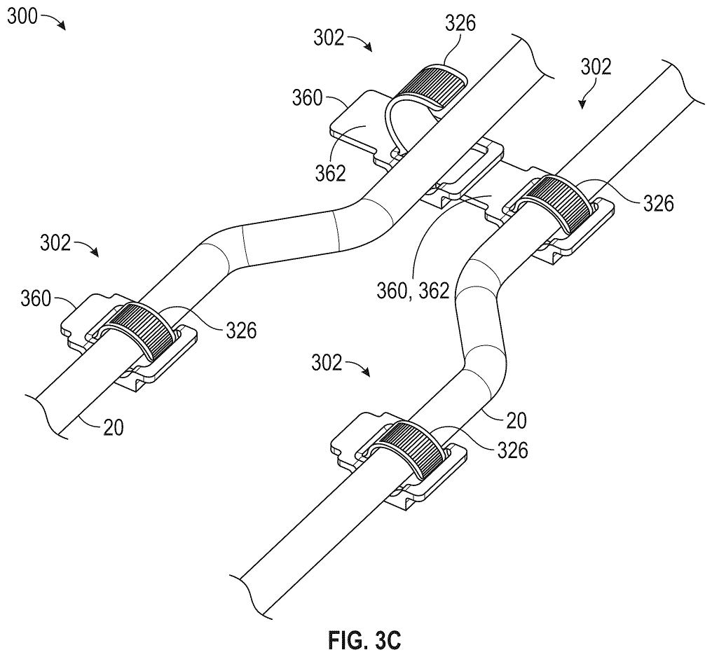

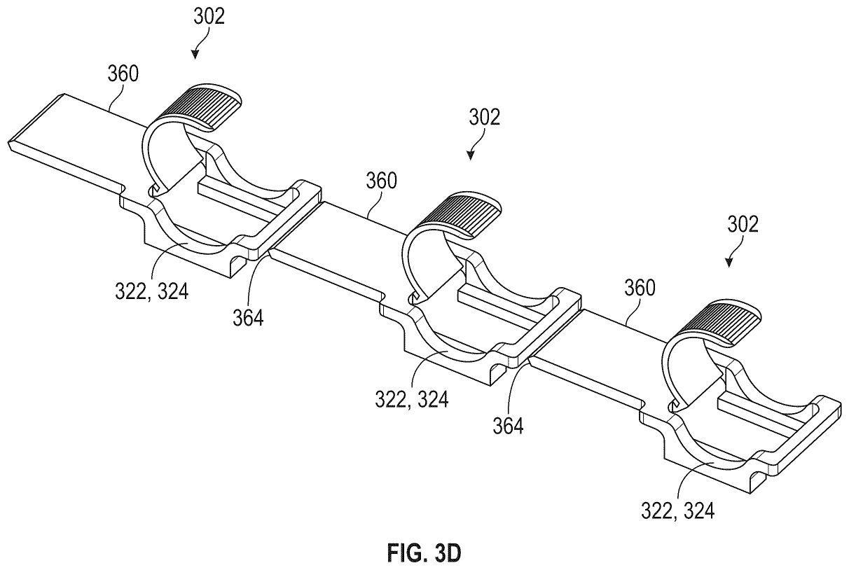

FIGS. 3A-3D illustrate a second example of the IV line attachment, in accordance with some embodiments disclosed herein. FIG. 3A shows the second example line attachment 302. The attachment mechanism of the line attachment 302 includes the receiving area 320 and the strap 326. As described in greater detail below with respect to FIG. 3C, the receiving area 320 is configured to receive an IV line, and the strap 326 is configured to secure the IV line to the top surface 322 of the receiving area 320. The strap 326 is coupled to a first side of the receiving area 320 by a hinge so that the strap 326 can pivot relative to the first side of the receiving area 320. In some embodiments, the hinge is a living hinge (i.e., the strap 326 and the receiving area 320 comprise a single component), which allows for simple and inexpensive manufacture of the line attachment 302.

FIG. 3C illustrates an IV line management system 300 with four line attachments 302 and two IV lines 20. As shown, the strap 326 is configured to be moved (e.g., by a caregiver) via the hinge to secure the IV line 20 to the receiving area 320 of the line attachment 302. In particular, the IV line 20 is strapped to the top surface 322 of the receiving area 320 by the strap 326. The free end of the strap 326 can connect to the second side of the receiving area 320, which is opposite the first side of the receiving area 320. In some embodiments, the second side of the receiving area 320 comprises a slot configured to receive the free end of the strap 326. In other embodiments, the strap 326 is configured to fit between the IV line 20 and an inner edge of the receiving area 320. Overall, the strap 326 and the receiving area 320 are designed for single-handed use. Optionally, the free end of the strap 326 is also designed to easily detach from the second side of the receiving area 320 to facilitate quick disconnection of the IV line 20 from the line attachment 326.

In the embodiments illustrated in FIGS. 3A-3D, the strap 326 of the line attachment 302 has one or more surfaces with ribs or ridges. This can also be referred to as a ratchet surface. In embodiments of the line attachment 302 where the strap 326 has a ratchet surface, the receiving area 320 can also include a pawl configured to engage with the ratchet surface. The pawl and the ratchet surface engage in a manner similar to that of a zip/cable tie or a ratchet mechanism. Accordingly, the pawl and the ratchet surface can resist movement or loosening of the strap 326 once the strap 326 is connected to the second side of the receiving area and, thus, limit movement (e.g., sliding, twisting, etc.) of the IV line relative to the line attachment 302. The pawl and the ratchet surface can also be used to prevent patients or other persons from tampering with the IV lines 20.

In some embodiments, the top surface 322 of the receiving area 320 comprises a groove or channel 324. This can be seen in FIG. 3D. The groove 324 has a curved profile that is shaped according to the contours of a standard IV line. A receiving area 320 with a groove 324 deforms the IV line less than a receiving area 320 with a flat top surface 322. The reduced deformation of the IV line limits the extent to which the line attachment 302 interferes with fluid flow through the IV line.

The line attachment 302 also has two coupling mechanisms in the form of the plate 360 and the rails 340. FIG. 3A depicts the plate 360 protruding from the first side of the receiving area 320 and the rails 340 protruding from the bottom surface 304 of the receiving area 320. Optionally, the plate 360 can protrude from the second side of the receiving area 320. The plate 360 is sized and shaped to slide into the rails 340, and the rails 340 form a shelf upon which the plate 360 can rest upon. FIG. 3B shows two line attachments 302 coupled together. The plates 360 and the rails 340 of the second example line attachments 302 all have the same designs and dimensions, which means that any two line attachments 302 can be connected via their plates 360 and rails 340.

FIG. 3C illustrates the line attachments 302 coupled to IV lines 20 as part of an IV line management system 300. As shown, a line attachment 302 can be connected to a neighboring line attachment 302 via the coupling mechanisms 340 and 360. Coupling the line attachments 302 together can provide benefits such as those described above with respect to the line attachments 202 and their coupling mechanisms (the protrusions 260 and the pockets 240).

FIG. 3D illustrates another type of coupling mechanism for the line attachment 302. In FIG. 3D, each of the line attachments 302 are semi-permanently connected by a perforation 364 between the second side of the receiving area 320 and the plate 360. A caregiver can either break the perforation 364 to separate each line attachment 302 and use the line attachments 302 in the manner described above, or the caregiver can use the perforations 364 to keep the line attachments 302 (and, thus, the IV lines) together. In some embodiments, the only coupling mechanism is the perforation 364, and the line attachment 302 does not have a plate 360 or rails 340.

Moreover, the line attachments 302 can also have indicators, such as those disclosed above with respect to the general description of the line attachments. For example, in FIG. 3C, two line attachments 302 are coupled to each IV line 20. The coordinated pairs of IV line attachments 302 (i.e., the line attachments 302 that are coupled to the same IV line 20) are depicted in FIG. 3C as being relatively close to each other on the IV line 20. In use, coordinated pairs of line attachments 302 can be placed on opposite ends of an IV line 20. For example, with reference to FIG. 1, one line attachment can be positioned downstream near the catheter 30 in the patient's arm 40 or near the patient's arm 40, and the other line attachment can be positioned upstream near the IV bag 10 or an infusion pump. Both the downstream and the upstream line attachments 302 have corresponding indicators, which is beneficial because a caregiver can gather information about any given IV line 20 (and its corresponding IV set) by looking at either end of the IV line 20.

In the embodiment of the line attachments 302 illustrated in FIG. 3C, the line attachments 302 can have different colors to serve as the indicators. In other embodiments, stickers or labels with indicating information (i.e., a colored stripe, a number, a letter, a QR code, etc.) can be adhered to the plate 360 of the line attachment 302. Optionally, the indicators can be numbers or letters that are engraved, embossed, or imprinted onto the plate 360.

In some embodiments of the line attachment 302, the plate 360 can comprise an application surface 362, which is a larger or extended version of the plate 360. In addition to being dimensioned to couple with the rails 340, the application surface 362 also provides the caregiver with enough space to record pertinent information about the fluids being delivered, such as the type of fluid, the time of administration, when the IV bag needs to be replaced, etc. Optionally, only one line attachment 302 on an IV line has an application surface 362. For instance, FIG. 3C shows two IV lines 20, each with two line attachments 302, but only one line attachment 302 on each IV line 20 has an application surface 362 on the plate 360. In use, the line attachment 302 with the application surface 362 can be placed near the IV bag so that a caregiver replacing the IV bag can readily view the information provided on the application surface 362. Accordingly, the line attachment 302 with the plate 360 can be placed on the portion of the IV line 20 that is closer to the patient to reduce the bulkiness of the IV set near the patient and, thus, reduce the discomfort experienced by the patient.

Additional features of the line attachment 302 that can help reduce the discomfort experienced by the patient are the rounded corners and the flexible material. As shown in FIGS. 3A-3D, the receiving area 320, the strap 326, and the protrusion 360 can all have rounded corners and edges. If the line attachment 302 is pressed against the patient, then the rounded corners and edges will reduce the amount of pain and discomfort experienced by the patient (as compared to traditionally sharp corners and edges). Similarly, the line attachment 302 can be made of a flexible material (such as rubber or silicon) that can (at least partially) conform to the patient when in contact with the patient instead of pressing into the patient's skin.

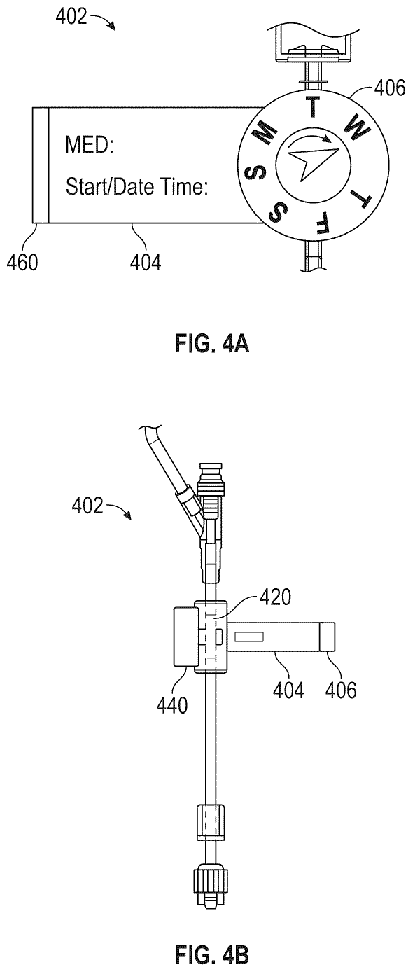

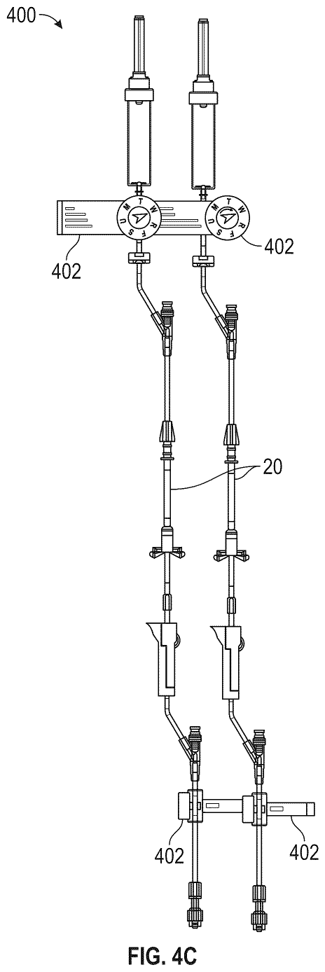

FIGS. 4A-4C illustrate a third example of the IV line attachment, in accordance with some embodiments disclosed herein. FIGS. 4A and 4B illustrate the third example IV line attachment 402. The attachment mechanism of the line attachment 402 is a C-clip or a groove (such as the groove 420 in FIG. 4B) on the line attachment 402.

The coupling mechanisms of the line attachment 402 are a protrusion 460 and a pocket 440, with the protrusion 460 and the pocket 440 being on opposite sides of the line attachment 402. The protrusion 460 and the pocket 440 are similar to the protrusion 260 and the pocket 240 described above with respect to the first example line attachment 202. Accordingly, the protrusion 460 can snap fit or slide into the pocket 440. Note that the pocket 440 of the line attachment 402 is not visible in FIG. 4A because it is obscured by the reminder dial 406, which will be discussed in greater detail below.

In some embodiments, the protrusion 460 can be a spherical or ball-shaped protrusion instead of a cylindrical protrusion, and the pocket 440 can be semi-spherical or socket-shaped. This embodiment would allow for more movement between the protrusion 460 and the pocket 440, which can limit disconnections between line attachments 402 in situations where the IV lines might be moved or jostled.

FIG. 4C depicts an IV line management system 400 with two IV lines 20 coupled together by line attachments 402. As described above, using the coupling mechanisms 440, 460 to keep the IV lines 20 in parallel (or fixed relative to each other) can help caregivers keep track of the fluids delivered to the patient via multiple IV sets.

The third example line attachment 402 is compatible with several types of indicators. In some embodiments, corresponding line attachments 402 (i.e., line attachments 402 that are on the same IV line 20) are the same color. In the embodiments shown in FIGS. 4A-4C, the protrusions 460 further comprise application surfaces 404, to which indicators, such as the ones described above, can be adhered, written, embossed, imprinted, etc. For example, a label or a sticker can be adhered to the application surface 404 to provide caregivers with information about the IV set.

In the embodiments depicted in FIGS. 4A-4C, both the downstream line attachment 402 and the upstream line attachment 402 have an application surface 404 for an indicator. This is beneficial because when both the line attachment 402 next to the patient and the line attachment 402 next to the IV bag have informational labels on the application surface 404, a caregiver can quickly and conveniently identify the fluid being transported by any given IV line 20.

The line attachment 402 also has a reminder dial 406, which is best seen in FIG. 4A. The reminder dial 406 can be manually operated or digital and shows the days of the week. The caregiver can use the reminder dial 406 to indicate an infusion start day or an infusion change day for the IV set to which the line attachment 402 is attached. Optionally, in a corresponding pair of line attachments 402, only the line attachment 402 that is near the IV bag has a reminder dial 406. This can reduce the size of the IV set near the patient, which makes being coupled to the IV set more comfortable for the patient.

FIGS. 5A-5E illustrate a fourth example of the IV line attachment, in accordance with some embodiments disclosed herein. FIG. 5A shows a perspective top view of the line attachment 502. As shown, the line attachment 502 has an attachment mechanism in the form of the groove 520, which is configured to receive an IV line. In some embodiments, the groove 520 is configured to snap fit around the IV line. In other embodiments, the groove 520 further comprises retaining clips that secure the IV line to the groove 520.

FIG. 5A also illustrates part of the coupling mechanism of the line attachment 502, which is the pocket 540. FIG. 5B, which is a perspective bottom view of the line attachment 502, illustrates the other part of the coupling mechanism, which is the protrusion 560. As shown, the pocket 540 and the protrusion 560 have corresponding ridges so that the protrusion 560 can fit inside the pocket 540. Optionally, the protrusion 560 can snap fit inside the pocket 540. FIGS. 5C and 5D show several line attachments 502 being coupled together via the protrusions 560 and the pockets 540.

FIGS. 5A and 5B illustrate the strap 580 that serves as the indicator for the line attachment 502. Each strap 580 wraps around the central brick or body of the line attachment 502. The strap 580 can be a solid color or display a pattern, number, QR code, bar code, or other label. In accordance with other examples of the line attachment described above, line attachments 502 in a coordinated pair have matching indicator straps 580. A caregiver can also adhere or otherwise attach a sticker or label to the strap 580, such as the stickers or labels described above.

FIG. 5B depicts a channel 506 on the bottom of the line attachment 502 because the line attachment 502 is configured to be secured to equipment in the vicinity of the patient and the IV sets, which can prevent the IV lines from moving excessively and, consequently, becoming tangled or dislodged. The channel 506 is depicted as having a curved profile, but other contours and shapes are also possible so that the line attachment 502 is compatible with a variety of rails, poles, and bars. For example, the line attachment 502 can be attached to a patient's bed rail or to an IV pole.

The strap 580 can wrap around an IV line, the body of the line attachment 502, and a rail, pole, or bar to secure the IV line to equipment such as the rail of a patient's bed. FIGS. 5A and 5B depict the holes 582 on the strap 580 and a notch 508 next to the channel 506. Any of the holes 582 can attach to the notch 508 to fit the strap 580 around different-sized rails, poles, or bars. In some embodiments, part of the strap 580 has hook and loop connectors so that the strap 580 can wrap around the rail, pole, or bar and hook onto itself. In other embodiments, the strap 580 is elastic and can stretch to fit around different-sized rails, poles, or bars.

Each example of the line attachments described herein is also compatible with a swing arm. A swing arm can include a spring base or a springboard configured to be placed on the ground. A pole extends from the base of the swing arm and contains pockets. Each of the pockets is configured to receive a line attachment.

### II. Intravenous Line Attachment Racks

Some embodiments of the IV line management system disclosed herein are directed to IV line attachment racks. Each line attachment rack has a plurality of attachment mechanisms by which IV lines can be coupled to the rack. In some embodiments, the line attachment rack can transition between an expanded configuration and a collapsed configuration. Optionally, the line attachment rack has additional attachment mechanisms when in the expanded configuration.

By using the line attachment rack, a caregiver can effectively connect several IV lines to organize and manage the IV lines. Not only can connecting the IV lines reduce the likelihood of the IV lines becoming tangled, but connecting the IV lines via the rack can also prevent the caregiver from mixing up the fluids associated with the IV lines. For example, with the IV lines connected to the rack, the caregiver can maintain a patient's IV lines in the exact same order, which reduces any ambiguity and confusion associated with identifying the fluid being delivered by each IV line. Additionally, a group of caregivers (e.g., nurses at a hospital who take turns taking care of the patient as they change shifts) can establish a standardized order for the IV lines and use the line attachment rack to maintain that standardized order. This can limit shuffling of the IV lines between various caregivers and reduce the likelihood that the incorrect fluid is delivered to the patient.

When using the IV line attachment racks as part of the IV line management system, each IV line will generally be attached to two racks: one rack near the IV bag (such as IV bag 10 in FIG. 1) and one rack closer to the patient. Optionally, an IV line can be attached to more than two racks (e.g., when an IV line is longer than average). This helps caregivers ensure that the proper fluid is being delivered to the patient (i.e., that the fluid from IV bag 10B in FIG. 1 is being delivered to the patient via IV line 20B and catheter 30B). Attachment mechanisms on the rack that are connected to the same IV line can be referred to as corresponding attachment mechanisms. Corresponding attachment mechanisms can be marked with matching indicators to signify that they are attached to the same IV line.

For example, referring to FIG. 1, IV line 20A can pass through two attachment mechanisms, each of which is on a separate line attachment rack, that are marked with a blue stripe. Similarly, IV line 20B can be connected to two different racks at two different attachment mechanisms, both of which have a red stripe as an indicator. A caregiver can use the indicators on any given IV line 20 to trace the IV line 20 between the IV bag 10 and the catheter 30 coupled to the patient. This can be useful because a busy caregiver can find it difficult to ensure that she is working with the correct IV set. With the indicators in place to help her quickly confirm, for instance, that the catheter 30B is connected to the IV bag 10B, the caregiver is less likely to remove catheter 30A from the patient's arm under the mistaken belief that catheter 30A is attached to IV bag 10B.

In some embodiments, the indicators are incorporated into the attachment mechanisms themselves. For example, corresponding attachment mechanisms can be the same color or be imprinted with the same number or letter. In other embodiments, the indicators are stickers or labels that can be applied to the line attachment racks. These indicators can comprise symbols, shapes, numbers, colors, QR codes, bar codes, etc. that are adhered to the outer surfaces of line attachments. Optionally, these indicators can comprise handwritten or typed labels with information about the type of medication, the infusion pump parameters, the medication administration times, etc.

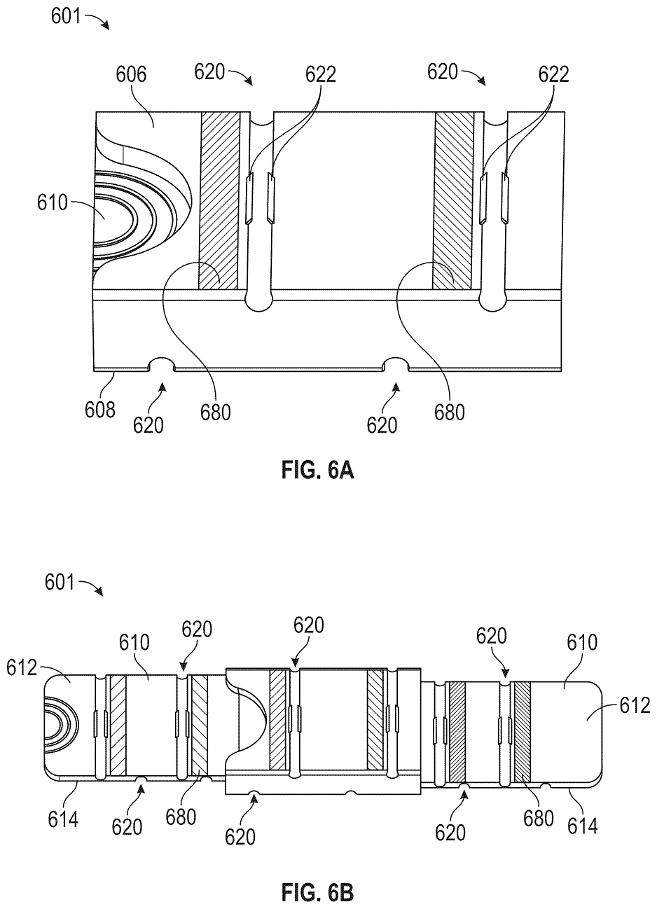

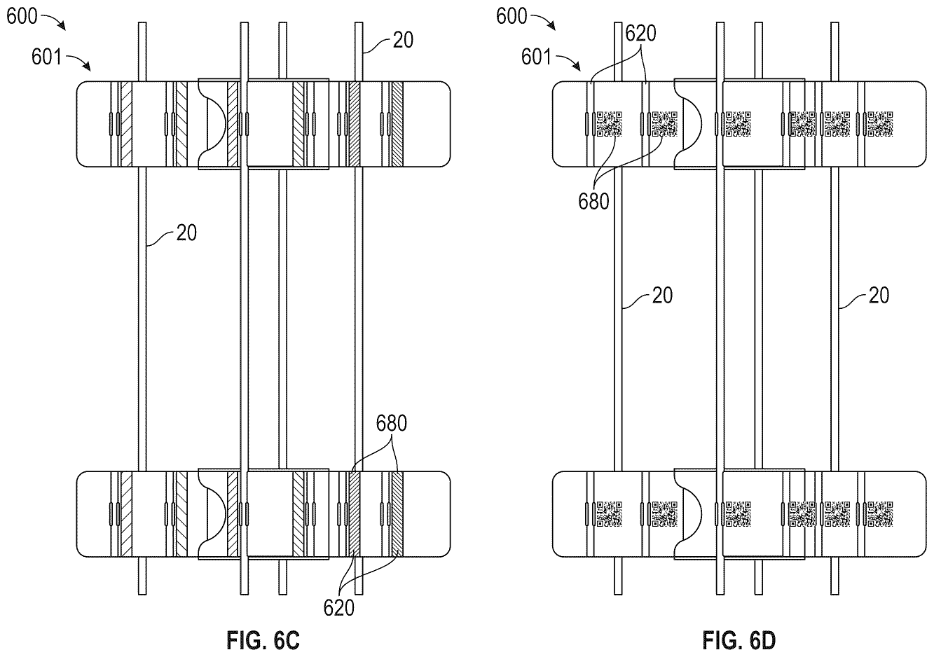

FIGS. 6A-6D illustrate a first example of the IV line attachment rack, in accordance with some embodiments disclosed herein. FIG. 6A depicts the first example of the IV line attachment rack 601 in a collapsed configuration. As shown, the rack 601 has a plurality of grooves 620 extending laterally across the top surface 606 of the rack 601. Each of the grooves 620 is configured to receive an IV line (such as one of the IV lines 20 in FIG. 1). In some embodiments, the grooves 620 have retaining clips 622 that can secure the IV line to the rack 601.

The rack 601 also has a bottom surface 608 and can include additional grooves 620 that extend laterally across the bottom surface 608 of the rack 601. Like the grooves 620 on the top surface 606, the grooves 620 on the bottom surface 608 can also have retaining clips 622.

FIG. 6A illustrates part of the sub-rack 610. The sub-rack 610 is slidably coupled to the rack 601 and can slide in and out of the rack 601 to transition the rack 601 between the collapsed configuration and the expanded configuration. When the rack 601 is in the collapsed configuration, the sub-rack 610 is stored within the rack 601, under the top surface 606. In the expanded configuration, which is shown in FIG. 6B, at least one sub-rack 610 extends longitudinally from the sides of the rack 601.

Like the main body of the rack 601, the sub-racks 610 have top surfaces 612 and bottom surfaces 614. One or both of the top and bottom surfaces 612, 614 can have grooves 620 for receiving additional IV lines. The grooves 620 can extend laterally across one or both sides of each sub-rack 610. Optionally, the grooves 620 on the sub-racks 610 can have retaining clips 622.

FIGS. 6C and 6D each illustrate an IV line management system 600. Each line management system 600 includes at least two line attachment racks 601 and IV lines 20. In use, the line attachment racks 601 can be coupled to opposite ends of the IV lines 20. For example, with reference to FIG. 1, one line attachment rack can be positioned downstream near the catheter 30 in the patient's arm 40 or near the patient's arm 40, and the other line attachment rack can be positioned upstream near the IV bag 10 or an infusion pump. Moreover, FIGS. 6C and 6D demonstrate that the IV lines 20 can be coupled to either side of the racks 601 and the sub-racks 610, which further helps organize the IV lines 20 by separating the IV lines 20 and minimizing tangling.

FIGS. 6C and 6D also depict two examples of indicators 680 on the racks 601. FIG. 6C shows that every groove 620 corresponds to a different colored stripe 680. Similarly, FIG. 6D illustrates that every groove 620 can correspond to a different QR code 680. Other indicators, such as the ones described above, are also possible. For example, the flat surfaces (of the top surfaces 606, 612 or bottom surfaces 608, 614) of the line attachment rack 601 and the sub-racks 610 can function as application surfaces (such as those described above with respect to the various example line attachments) to which labels, stickers, or other indicators can be applied.

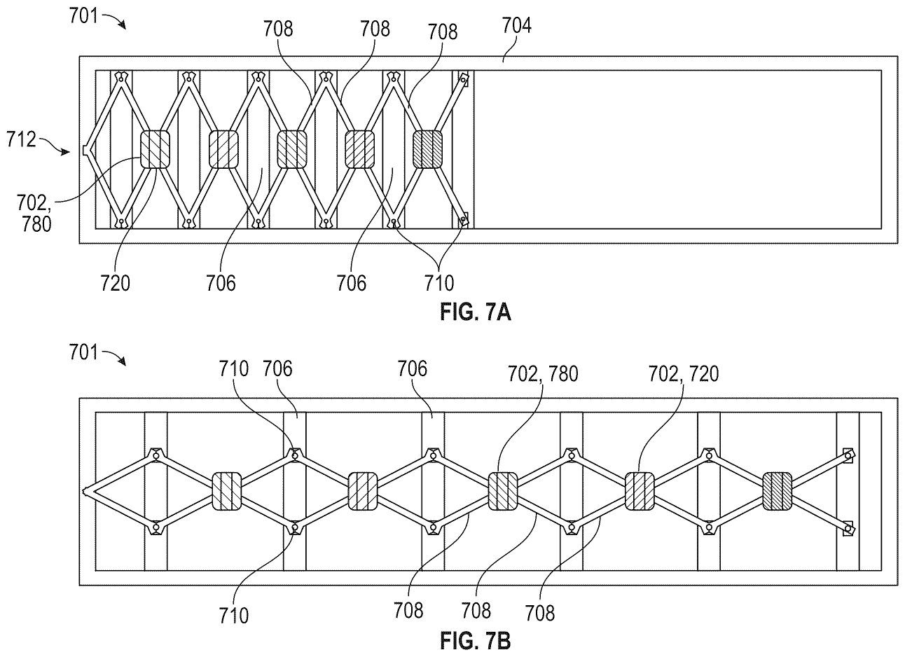

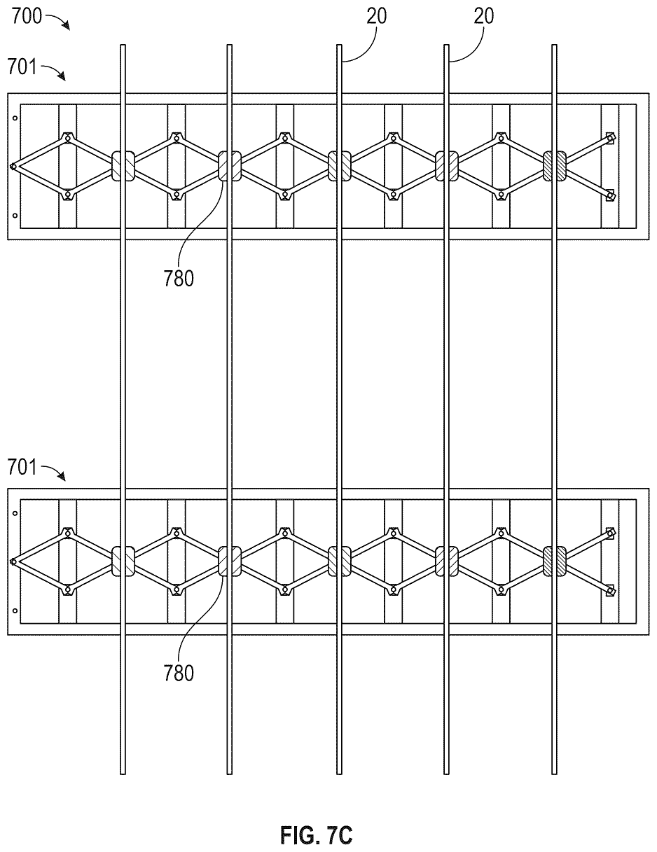

FIGS. 7A-7C illustrate a second example of the IV line attachment rack, in accordance with some embodiments disclosed herein. FIG. 7A illustrates the second example of the IV line attachment rack 701, which includes a plurality of line attachments 702 that have grooves 720. Like the grooves described above with respect to the various examples of the IV line attachment mechanisms, each groove 720 is configured to receive an IV line. In some embodiments, each line attachments 702 is coupled to two forks 708 by a joint (not shown) such that the forks 708 can rotate relative to the line attachment 702. In other embodiments, each line attachment 702 is coupled to only one fork 708. This can be done by connecting the line attachment 702 to the fork 708 using a joint, by adhering the line attachment 702 to the fork 708, or by otherwise fastening the line attachment 702 to the fork 708.

The rack 701 is configured to collapse and expand via a connected structure made of a frame 704, columns 706, forks 708, and pivots 710, 712. In the collapsed configuration, shown in FIG. 7A, the columns 706 are closer together, the forks 708 have a steeper slope, and the line attachments 702 are closer together (as compared to the expanded configuration). On the other hand, in the expanded configuration, shown in FIG. 7B, the columns 706 are spread apart, the forks 708 are in a more horizontal position, and the line attachments 702 are spread apart. In the expanded configuration, a caregiver can more easily distinguish between individual IV lines that are coupled to the line attachments 702 of the rack 701 (as shown in FIG. 7C).

In the embodiment shown in FIGS. 7A-7C, the columns 706 slide relative to the frame 704 to transition the rack 701 between the collapsed and expanded configurations. The columns 706 can sit within a track formed by the frame 704. Each column 706 is coupled to adjacent columns 706 by forks 708. The forks 708 are connected to the columns 706 by joints 710. The joints 710 are configured to slide up and down the column 706 as the rack 701 transitions between the collapsed configuration in FIG. 7A and the expanded configuration in FIG. 7B. The forks 708 can also rotate about the joints 710 as the rack 701 collapses and expands. Moreover, at least one of the right-most or the left-most columns 706 are coupled to the frame 704 by forks 708. In the embodiments of the line attachment rack 701 shown in FIGS. 7A-7C, the left-most column 706 is coupled to the frame 704 by two forks 708. The forks 708 are connected to the frame 704 by a joint 712. The forks 708 can rotate about the joint 712 as the columns 706 slide along the frame 704.

In some embodiments, the frame 704 itself can collapse and expand. In those embodiments, the columns 706 can be fixed relative to the frame 704, and collapsing the frame 704 can cause each column 706 to move relative to the other columns 706. The columns 706 are connected to each other by forks 708, and the forks 708 are coupled to the columns by joints 710. As discussed above with respect to the previous embodiment of the rack 701, as the rack 701 transitions between the collapsed and expanded configurations, the joints 710 can translate up and down the columns 706, and the forks 708 can rotate about the joints 710.

FIG. 7C depicts an IV line management system 700, which includes at least two IV line attachment racks 701 and several IV lines 20. The embodiment of the rack 701 shown in FIG. 7C has indicators in the form of different colored 780 line attachments 702. As shown, each line attachment 702 that is connected to the same IV line 20 has the same color 780. Accordingly, a caregiver can trace an IV line 20 by identifying the color of the line attachment 702 to which the IV line 20 is connected. Other indicators, such as the ones described above, are also compatible with the rack 701.

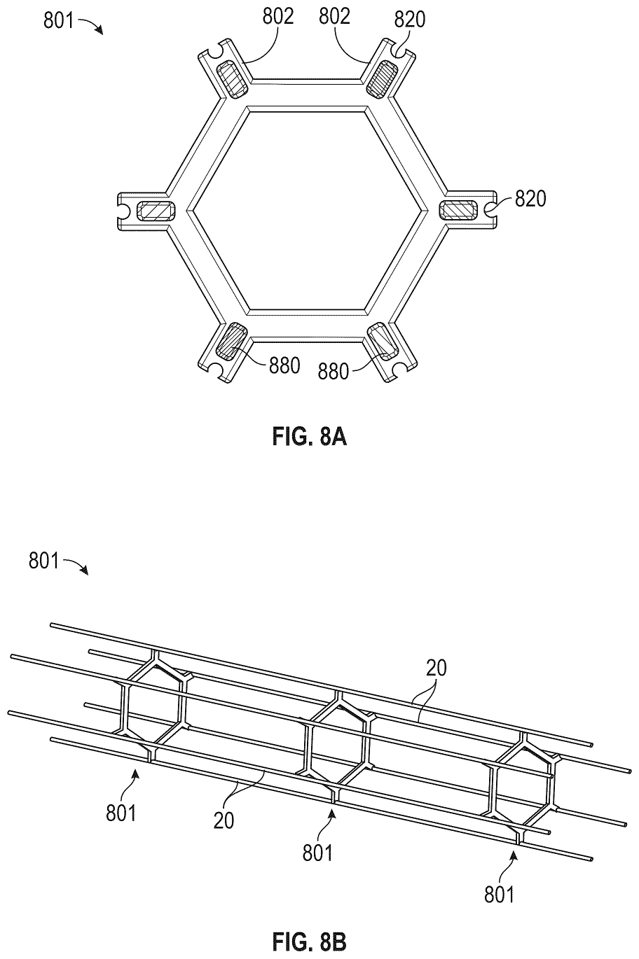

FIGS. 8A and 8B illustrate a third example of the IV line attachment rack, in accordance with some embodiments disclosed herein. FIG. 8A illustrates the third example of the IV line attachment rack 801. The rack 801 has a plurality of line attachments 802, and each line attachment 802 has a groove 820, such as the grooves that constitute the attachment mechanisms of the line attachments and line attachment racks described above. That is, each groove 820 is configured to receive an IV line.

The rack 801 is a polygonal ring or frame that has line attachments 802 positioned on the perimeter of the ring or frame. In the embodiment shown in FIGS. 8A and 8B, the rack 801 is a hexagonal frame with line attachments 802 positioned at each corner of the hexagon. The frame structure of the rack 801 facilitates separation of each of the line attachments 802, which spreads out the IV lines connected to the line attachments 802 and reduces the likelihood of the IV lines becoming tangled or twisted. In some embodiments, the frame or ring of the rack 801 can collapse, which can be beneficial for efficient storage of the rack 801 or for use in smaller spaces.

FIG. 8B illustrates an IV line management system 800 with three racks 801 connected to IV lines 20. As shown, each IV line 20 is connected to three racks 801 via three corresponding line attachments 802. FIG. 8A shows that each line attachment 802 of the rack 801 has an indicator 880. In FIG. 8A, the indicator 880 is a different colored marker. The colored indicators 880 can have fluorescent coatings that facilitate higher visibility, which is beneficial in low light settings (e.g., a caregiver replacing IV bags at night). The colored indicators 880 can be painted on, be built into the frame of the rack 801, or be stickers adhered to the frame. Other indicators (such as the ones described above with respect to the various examples of the line attachment and the line attachment racks) are also compatible with the rack 801. For example, the rack 801 can have QR codes in place of the colored indicators 880.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 9, or clause 16. The other clauses can be presented in a similar manner.

Clause 1. A management system for intravenous (IV) lines comprising at least one pair of line attachments, each line attachment of the at least one pair of line attachments being configured to attach to one of the IV lines and comprising: an inner hose comprising (a) a tube configured to receive the IV line and (b) a slot extending longitudinally along the tube, the inner hose being configured to flex open at the slot and wrap around the IV line; and an outer housing comprising (a) a hollow interior configured to receive the inner hose and (b) an outer surface comprising a protrusion and a pocket, wherein the protrusion of each line attachment is configured to be removably coupled to the pocket of another line attachment, and the pocket of each line attachment is configured to removably coupled to the protrusion of another line attachment.

Clause 2. The management system of Clause 1, wherein a first pair of line attachments is configured to attach to a first IV line of the IV lines and a second pair of line attachments is configured to attach to a second IV line of the IV lines.

Clause 3. The management system of Clause 2, wherein both line attachments in the first pair of line attachments comprises a first color and both line attachments in the second pair of line attachments comprises a second color, the first color being different from the second color.

Clause 4. The management system of Clause 2, wherein both line attachments in the first pair of line attachments comprises a first QR code and both line attachments in the second pair of line attachments comprises a second QR code, the first QR code being different from the second QR code.

Clause 5. The management system of Clause 1, wherein the inner hose comprises one or more indicators, and the outer housing comprises a window configured to surround one indicator of the one or more indicators when the inner hose is within the hollow interior of the outer housing.

Clause 6. The management system of Clause 1, wherein the protrusion of each line attachment comprises an application surface.

Clause 7. The management system of Clause 1, wherein a first line attachment of the at least one pair of line attachments is configured to be positioned next to a fluid source, and a second line attachment of the at least one pair of line attachments is configured to be positioned next to a patient receiving fluid from the fluid source.

Clause 8. The management system of Clause 1, wherein each line attachment comprises a capacitive sensor configured to identify a fluid in the IV line when the line attachment is attached to the IV line.

Clause 9. A management system for intravenous (IV) lines comprising at least one pair of line attachments, each line attachment of the at least one pair of line attachments being configured to attach to one of the IV lines, each line attachment comprising: a receiving area comprising a top surface configured to receive the IV line, a bottom surface, a first side, and a second side opposite the first side; a strap movably coupled to a first side of the receiving area by a hinge, wherein the strap is configured to connect to the second side of the receiving area to couple the IV line to the line attachment when the IV line is in the receiving area; a plate protruding from either the first side or the second side of the receiving area; and rails protruding from the bottom surface of the receiving area, wherein the plate of each line attachment is configured to be removably coupled to the rails of another line attachment, and the rails of each line attachment is configured to removably coupled to the plate of another line attachment.

Clause 10. The management system of Clause 9, wherein the top surface of the receiving area comprises a groove, wherein the groove is configured to limit deformation of the IV line when the IV line is coupled to the line attachment by the strap.

Clause 11. The management system of Clause 9, wherein the strap comprises a ratchet surface and the second side of the receiving area comprises a pawl, the ratchet surface being configured to secure the IV line to the receiving area by engaging with the pawl.

Clause 12. The management system of Clause 9, wherein a first pair of line attachments is configured to attach to a first IV line of the IV lines and a second pair of line attachments is configured to attach to a second IV line of the IV lines.

Clause 13. The management system of Clause 12, wherein both line attachments in the first pair of line attachments comprises a first color and both line attachments in the second pair of line attachments comprises a second color, the first color being different from the second color.

Clause 14. The management system of Clause 12, wherein both line attachments in the first pair of line attachments comprises a first QR code and both line attachments in the second pair of line attachments comprises a second QR code, the first QR code being different from the second QR code.

Clause 15. The management system of Clause 9, wherein at least one line attachment in each pair of line attachments comprises a near field radio frequency identification (RFID) tag.

Clause 16. A management system for intravenous (IV) lines comprising line attachment racks configured to receive the IV lines, each rack comprising: a plurality of grooves, each groove of the plurality of grooves being configured to receive one IV line of the IV lines; and at least one sub-rack slidably coupled to the rack, wherein the at least one sub-rack is positioned inside the rack when the rack is in a collapsed configuration and extends longitudinally from the rack when the rack is in an expanded configuration, wherein a first portion of grooves of the plurality of grooves extends laterally across a top surface of the rack and a second portion of the plurality of grooves extends laterally across a top surface the at least one sub-rack.

Clause 17. The management system of Clause 16, wherein the line attachment racks comprise a first rack and a second rack, and each groove on the first rack corresponds with another groove on the second rack, such that corresponding grooves are configured to receive a same IV line.

Clause 18. The management system of Clause 17, further comprising a QR code or a color indicator adjacent to each groove, wherein the corresponding grooves each comprise matching QR codes or color indicators.

Clause 19. The management system of Clause 16, wherein a third portion of grooves of the plurality of grooves extends laterally across a bottom surface of the rack and a fourth portion of the plurality of grooves extends laterally across a bottom surface of the at least one sub-rack.

Clause 20. The management system of Clause 16, wherein the at least one sub-rack comprises a first sub-rack and a second sub-rack, wherein the first sub-rack is positioned inside the rack adjacent to the top surface of the rack when the rack is in the collapsed configuration and extends longitudinally from a first side of the rack when the rack is in the expanded configuration, wherein the second sub-rack is positioned inside the rack adjacent to a bottom surface of the rack when the rack is in the collapsed configuration and extends longitudinally from a second side of the rack when the rack is in the expanded configuration, the second side of the rack being opposite the first side.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear,” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A management system for intravenous (IV) lines comprising at least one pair of line attachments,
each line attachment of the at least one pair of line attachments being configured to attach to one of the IV lines and comprising:
an inner hose comprising (a) a tube configured to receive the IV line and (b) a slot extending longitudinally along the tube, the inner hose being configured to flex open at the slot and wrap around the IV line; and
an outer housing comprising (a) a hollow interior configured to receive the inner hose and (b) an outer surface comprising a protrusion and a pocket,
wherein the protrusion of each line attachment is configured to be removably coupled to the pocket of another line attachment, and the pocket of each line attachment is configured to removably coupled to the protrusion of another line attachment.

2. The management system of claim 1, wherein a first pair of line attachments is configured to attach to a first IV line of the IV lines and a second pair of line attachments is configured to attach to a second IV line of the IV lines.

3. The management system of claim 2, wherein both line attachments in the first pair of line attachments comprises a first color and both line attachments in the second pair of line attachments comprises a second color, the first color being different from the second color.

4. The management system of claim 2, wherein both line attachments in the first pair of line attachments comprises a first QR code and both line attachments in the second pair of line attachments comprises a second QR code, the first QR code being different from the second QR code.

5. The management system of claim 1, wherein the inner hose comprises one or more indicators, and the outer housing comprises a window configured to surround one indicator of the one or more indicators when the inner hose is within the hollow interior of the outer housing.

6. The management system of claim 1, wherein the protrusion of each line attachment comprises an application surface.

7. The management system of claim 1, wherein a first line attachment of the at least one pair of line attachments is configured to be positioned next to a fluid source, and a second line attachment of the at least one pair of line attachments is configured to be positioned next to a patient receiving fluid from the fluid source.

8. The management system of claim 1, wherein each line attachment comprises a capacitive sensor configured to identify a fluid in the IV line when the line attachment is attached to the IV line.

9. A management system for intravenous (IV) lines comprising at least one pair of line attachments,
each line attachment of the at least one pair of line attachments being configured to attach to one of the IV lines, each line attachment comprising:
a receiving area comprising a top surface configured to receive the IV line, a bottom surface, a first side, and a second side opposite the first side;
a strap movably coupled to a first side of the receiving area by a hinge, wherein the strap is configured to connect to the second side of the receiving area to couple the IV line to the line attachment when the IV line is in the receiving area;
a plate protruding from either the first side or the second side of the receiving area; and
rails protruding from the bottom surface of the receiving area,
wherein the plate of each line attachment is configured to be removably coupled to the rails of another line attachment, and the rails of each line attachment is configured to removably coupled to the plate of another line attachment.

10. The management system of claim 9, wherein the top surface of the receiving area comprises a groove, wherein the groove is configured to limit deformation of the IV line when the IV line is coupled to the line attachment by the strap.

11. The management system of claim 9, wherein the strap comprises a ratchet surface and the second side of the receiving area comprises a pawl, the ratchet surface being configured to secure the IV line to the receiving area by engaging with the pawl.

12. The management system of claim 9, wherein a first pair of line attachments is configured to attach to a first IV line of the IV lines and a second pair of line attachments is configured to attach to a second IV line of the IV lines.

13. The management system of claim 12, wherein both line attachments in the first pair of line attachments comprises a first color and both line attachments in the second pair of line attachments comprises a second color, the first color being different from the second color.

14. The management system of claim 12, wherein both line attachments in the first pair of line attachments comprises a first QR code and both line attachments in the second pair of line attachments comprises a second QR code, the first QR code being different from the second QR code.

15. The management system of claim 9, wherein at least one line attachment in each pair of line attachments comprises a near field radio frequency identification (RFID) tag.

16. A management system for intravenous (IV) lines comprising line attachment racks configured to receive the IV lines, each rack comprising:
a plurality of grooves, each groove of the plurality of grooves being configured to receive one IV line of the IV lines; and
at least one sub-rack slidably coupled to the rack, wherein the at least one sub-rack is positioned inside the rack when the rack is in a collapsed configuration and extends longitudinally from the rack when the rack is in an expanded configuration,
wherein a first portion of grooves of the plurality of grooves extends laterally across a top surface of the rack and a second portion of the plurality of grooves extends laterally across a top surface the at least one sub-rack.

17. The management system of claim 16, wherein the line attachment racks comprise a first rack and a second rack, and each groove on the first rack corresponds with another groove on the second rack, such that corresponding grooves are configured to receive a same IV line.

18. The management system of claim 17, further comprising a QR code or a color indicator adjacent to each groove, wherein the corresponding grooves each comprise matching QR codes or color indicators.

19. The management system of claim 16, wherein a third portion of grooves of the plurality of grooves extends laterally across a bottom surface of the rack and a fourth portion of the plurality of grooves extends laterally across a bottom surface of the at least one sub-rack.

20. The management system of claim 16, wherein the at least one sub-rack comprises a first sub-rack and a second sub-rack,
wherein the first sub-rack is positioned inside the rack adjacent to the top surface of the rack when the rack is in the collapsed configuration and extends longitudinally from a first side of the rack when the rack is in the expanded configuration,
wherein the second sub-rack is positioned inside the rack adjacent to a bottom surface of the rack when the rack is in the collapsed configuration and extends longitudinally from a second side of the rack when the rack is in the expanded configuration, the second side of the rack being opposite the first side.
